(19) United States
(12) Patent Application Publication
(10) Pub. No.: US 2025/0248607 A1
(43) Pub. Date: Aug. 7, 2025

(54) APPARATUS FOR QUICK DETECTION

(71) Applicants: TING-JU CHEN, Hsinchu City (TW); YUN-CHIEN WANG, Hsinchu City (TW); HENG-YI LIN, New Taipei City (TW)

(72) Inventors: TING-JU CHEN, Hsinchu City (TW); YUN-CHIEN WANG, Hsinchu City (TW)

(73) Assignees: TING-JU CHEN, Hsinchu City (TW); YUN-CHIEN WANG, Hsinchu City (TW); HENG-YI LIN, New Taipei City (TW)

(21) Appl. No.: 18/429,721

(22) Filed: Feb. 1, 2024

(51) Int. Cl. A61B 5/021; A61B 5/00; A61B 7/04

(52) U.S. Cl. CPC A61B 5/021; A61B 5/7267; A61B 5/743; A61B 7/04

(57) ABSTRACT

An apparatus for quick detection of a blood pressure level is described. The apparatus comprises a module, a device and a network processor. The module is for capturing data of a heart sound. The device is for transforming the data into a plurality of time-frequency spectrograms having a plurality of image features. The network processor is trained to analyze the image features of the time-frequency spectrograms, for giving the blood pressure level. The network processor is equipped with a convolutional neural network trained to analyze the image features of the time-frequency spectrograms, by focusing on a second heart sound, for detecting the hypertension. The network processor is also trained to identify the time-frequency spectrograms within a frequency band, to distinguish the long-term hypertension and the exercise-induced transient hypertension.

### PRIOR PUBLIC DISCLOSURE OF THE INVENTION

The invention described herein was previously publicly disclosed in accordance with 35 U.S.C. § 102 (b)(1). Specifically, the invention was exhibited at the “Taiwan International Science Exhibition 2023”, held in Taipei, Taiwan, from February 6 to February 10. The exhibition was a public event and the features of the invention were disclosed to the attendees without any confidentiality restrictions. The disclosed invention included “Deep Learning Study on Heart Sound and Hypertension Association”, which aligns with the embodiment of the invention as described in the present application. Moreover, the invention was also exhibited at the “Young people and science 2023”, held in Milan, Italy, from March 18 to March 20. The exhibition was a public event and the features of the invention were disclosed to the attendees without any confidentiality restrictions. The disclosed invention included “Using CNN Model for Hypertension Detection with Heart Sound”, which aligns with the embodiment of the invention as described in the present application. The public disclosure during the aforementioned exhibition does not bar this application from being granted a patent as it falls within the one-year grace period provided by the United States patent law. The present patent application is being filed within one year from the initial public disclosure date, which is within the statutory period allowed for preserving the patent rights post-disclosure.

### FIELD

The present invention relates to an apparatus, and, more specifically, to an apparatus for quick detection.

### BACKGROUND

According to the World Health Organization, 1.28 billion adults are living with hypertension, yet 46% of them remain unaware. The standard method for measuring blood pressure involves sphygmomanometers, which necessitate pressure on the arm, making it unsuitable for individuals with arm injuries. Additionally, a sudden spike in blood pressure may lead to urgent and severe health conditions. Hence, there is a need to develop a non-pressurized, continuous blood pressure detection apparatus.

### SUMMARY

According to at least one exemplary embodiment, an apparatus for quick detection may be shown and described. The apparatus may comprise a module, a device, and a network processor. The module may be for capturing data of a heart sound. The device may be for transforming the data into a plurality of time-frequency spectrograms having a plurality of image features. The network processor may be trained to analyze the image features of the time-frequency spectrograms. The image features may be analyzed for giving a blood pressure level. The apparatus may further comprise an output interface for displaying the blood pressure level.

Alternatively, the apparatus may comprise a non-pressurized module, a device, and a network processor. The non-pressurized module may be for capturing data of a second heart sound. The device may be for transforming the data into a plurality of time-frequency spectrograms. The network processor may be equipped with a convolutional neural network trained to analyze the image features of the time-frequency spectrograms, by focusing on the second heart sound. The image features may be analyzed for detecting hypertension.

Each of the time-frequency spectrograms may comprise a first frequency band and a second frequency band. The network processor may be trained to analyze the image features of the time-frequency spectrograms within the first frequency band and the second frequency band. The first frequency band may be about 0 Hz to about 200 Hz. The second frequency band may be about 400 Hz to about 600 Hz. The non-pressurized module may capture the data by recording the second heart sound for about 20 to about 30 seconds. The second heart sound may be recorded from an individual who has been sitting down for a plurality of minutes. The second heart sound may be recorded from the individual who has been sitting down for about five minutes. The second heart sound may be recorded from the individual who has exercised. The second heart sound may be recorded from the individual who has exercised to have a heart rate of about 120 beats per minute.

In another exemplary embodiment, an apparatus for quick detection is described to comprise a module, a device and a network processor. The module may be for capturing data of a heart sound. The device may be for transforming the data into a plurality of time-frequency spectrograms having a plurality of image features. Each of the time-frequency spectrograms may comprise a frequency band. The network processor may be trained to identify the time-frequency spectrograms within the frequency band, to distinguish long-term hypertension and exercise-induced transient hypertension.

The frequency band may be about 0 Hz to about 200 Hz. Alternatively, the frequency band may be about 400 Hz to about 600 Hz. The module may capture the data by recording the heart sound for about 20 to about 30 seconds. The heart sound may be recorded from an individual who has been sitting down for a plurality of minutes. The heart sound may be alternatively recorded from the individual who has exercised to have a heart rate of about 120 beats per minute.

### BRIEF DESCRIPTION OF THE DRAWINGS

FIG. 1 illustrates, according to a first embodiment of the present invention, a schematic diagram of an apparatus for quick detection.

FIG. 2 illustrates, according to a second embodiment of the present invention, a schematic diagram of an apparatus for quick detection.

FIG. 3 illustrates, according to a third embodiment of the present invention, a schematic diagram of an apparatus for quick detection.

FIG. 4 illustrates a training procedure of a network processor.

FIG. 5 shows a flowchart of the training procedure.

FIG. 6 is a Loss graph for four-layer model training with S2 data.

FIG. 7 is an accuracy graph for four-layer model training with S2 data.

FIG. 8 is a schematic diagram of the confusion matrix.

FIG. 9 is a diagram that presents the performance of models trained with S1+S2 Dataset and S2 Dataset

FIG. 10 shows a diagram that presents the performance of models trained by different frequency bands.

FIG. 11 is the confusion matrix for the model training distinguishing between post-exercise hypertension and long-term hypertension.

FIG. 12 schematically illustrates the time-frequency spectrograms transformed by a device.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

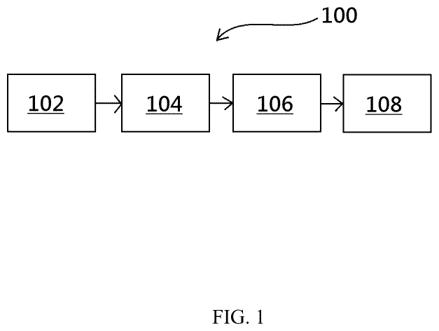

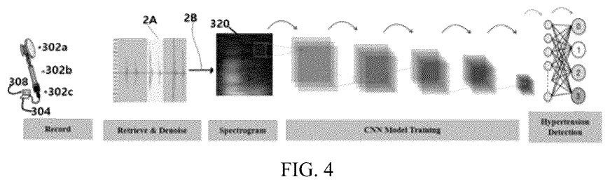

In a first embodiment, apparatus for quick detection is described. Referring to FIG. 1, the apparatus 100 may comprise a module 102, a device 104, and a network processor 106. The module 102 may be for capturing data of a heart sound. The module may comprise a chest piece 302a (FIG. 4) of a stethoscope, a microphone 302c and a catheter 302b between the microphone 302c and the chest piece 302a of the stethoscope. The device 104 may be a computer for transforming the data into a plurality of time-frequency spectrograms having a plurality of image features.

The data may be transformed by a technique of Short-time Fourier Transform. The Short-time Fourier Transform (STFT) is a derivative technique based on the Fourier Transform. It divides a long-duration sound signal into several shorter and equal-length signals, and then computes the Fourier Transform for these shorter durations. It's an important tool in time-frequency analysis.

This transformation can convert a segment of sound signal into a time-frequency spectrogram that records the frequency and amplitude of sound over time. In the spectrogram, the frequency components are represented on the vertical axis, time on the horizontal axis, and the intensity of sound is indicated by varying brightness.

When using the Short-time Fourier Transform, one can adjust the length of the window to determine the time resolution and frequency resolution. A longer window length captures a longer sound signal, resulting in higher frequency resolution but lower time resolution, and vice versa. Thus, there is a trade-off between time resolution and frequency resolution, and one must choose the most suitable window length based on their requirements.

The calculation method of the Short-time Fourier Transform involves multiplying a function by a window function and then performing a one-dimensional Fourier Transform. The window function shifts over time, yielding a series of Fourier Transform results, which are then arranged into a two-dimensional representation. The mathematical definition can be expressed as:

$$X ( t , f ) = {\int }_{- ∞}^{∞} w ( t - τ ) x ( τ ) e - j 2 π f τ d τ$$

In the above formula, w(t) represents the window function, and x(t) is the signal to be analyzed.

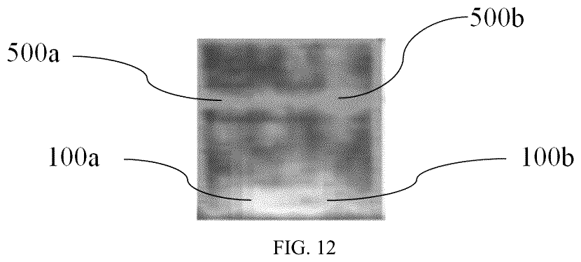

The term “image feature” may refer to any change in brightness or color that appears within a plurality of time-frequency spectrograms. FIG. 12 schematically illustrates the time-frequency spectrograms transformed by the device 104. Referring to FIG. 12, a plurality of image feature 100a, 100b, 500a and 500b, i.e., changes in brightness or color that appears within the time-frequency spectrograms.

Referring to FIG. 12 and FIG. 1, the network processor 106 may be trained to analyze the image features 100a, 100b, 500a and 500b of the time-frequency spectrograms. The “network” processor 106 is capable of employing “Convolutional Neural Networks” (CNNs) for computations, facilitating deep learning processes. This capability allows the processor 106 to be trained to analyze image features 100a, 100b, 500a, and 500b of the time-frequency spectrograms. CNNs, an algorithm central to deep learning, exhibit an architecture that mirrors the neuronal connectivity patterns observed in the human brain, particularly within the visual cortex. Their extensive application in image recognition stems from their proficiency in parameter reduction through weight sharing and the preservation of image characteristics via convolutional, pooling, and fully connected layers. The number and configuration of these layers in a CNN significantly influence the training results, leading to variations in the model's performance and adaptations in subsequent implementations.

Referring to FIG. 12 and FIG. 1, the image features 100a, 100b, 500a, and 500b may be analyzed for giving a blood pressure level. The apparatus 100 may further comprise an output interface 108 for displaying the blood pressure level.

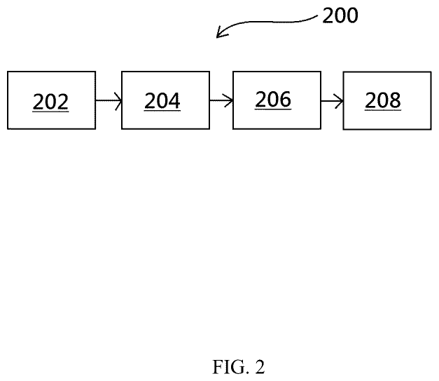

In a second embodiment, an apparatus for quick detection is described. Referring to FIG. 2, the apparatus 200 may comprise a non-pressurized module 202, a device 204, and a network processor 206. The non-pressurized module 202 may be for capturing data of a second heart sound (S2). The device 204 may be for transforming the data into a plurality of time-frequency spectrograms. The network processor 206 may be equipped with a convolutional neural network trained to analyze the image features of the time-frequency spectrograms, by focusing on the second heart sound (S2). The image features may be analyzed for detecting hypertension.

In the second embodiment, each of the time-frequency spectrograms may comprise a first frequency band and a second frequency band. The network processor 206 may be trained to analyze the image features of the time-frequency spectrograms within the first frequency band and the second frequency band. The first frequency band may be about 0 Hz to about 200 Hz. The second frequency band may be about 400 Hz to about 600 Hz. The non-pressurized module 202 may capture the data by recording the second heart sound (S2) for about 20 to about 30 seconds. The second heart sound (S2) may be recorded from an individual who has been sitting down for a plurality of minutes. The second heart sound (S2) may be recorded from the individual who has been sitting down for about five minutes. The second heart sound (S2) may be recorded from the individual who has exercised. The second heart sound (S2) may be recorded from the individual who has exercised to have a heart rate of about 120 beats per minute.

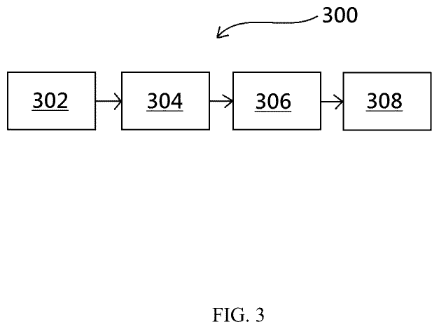

In a third embodiment, an apparatus for quick detection is described. Referring to FIG. 3, an apparatus 300 for quick detection is described to comprise a module 302, a device 304 and a network processor 306. The module 302 may be for capturing data of a heart sound. The device 304 may be for transforming the data into a plurality of time-frequency spectrograms having a plurality of image features. Each of the time-frequency spectrograms may comprise a frequency band. The network processor 306 may be trained to identify the time-frequency spectrograms within the frequency band, to distinguish long-term hypertension and exercise-induced transient hypertension.

In the third embodiment, the frequency band may be about 0 Hz to about 200 Hz. Alternatively, the frequency band may be about 400 Hz to about 600 Hz. The module may capture the data by recording the heart sound for about 20 to about 30 seconds. The heart sound may be recorded from an individual who has been sitting down for a plurality of minutes. The heart sound may be alternatively recorded from the individual who has exercised to have a heart rate of about 120 beats per minute.

Convolutional Neural Network (CNN) is an algorithm in the field of deep learning. Its architecture is similar to the connection patterns of neurons in the human brain, with inspiration coming from the organization of the visual cortex. CNNs are extensively applied in image recognition due to their ability to reduce the number of parameters through shared weights and maintain image features using convolutional layers, pooling layers, and fully connected layers. The number of these layers in the model can lead to different training outcomes; hence, subsequent embodiments will also adjust these aspects accordingly.

Hypertension has various causes and can be divided into two main types: primary hypertension and secondary hypertension. Primary hypertension is high blood pressure with no identifiable cause and may be attributed to genetics, diet, stress, lack of exercise, among other reasons. Secondary hypertension occurs due to specific causes such as kidney disease, congenital arterial abnormalities, endocrine disorders, or side effects of certain medications. The aforementioned types of hypertension belong to a condition of persistently high blood pressure; however, exercise can also cause a temporary increase in blood pressure. During physical activity, muscles demand more oxygen, which activates the sympathetic nervous system and increases the heart rate to augment cardiac output. As both the heart rate and the stroke volume increase, blood pressure rises accordingly.

The following examples are provided for illustrative purposes to demonstrate the applications of the disclosed invention to enable a quick determination of a blood pressure level, and detection of hypertension. These examples do not impose limitations on the broader scope of the disclosed invention.

### (I) Measuring Equipment:

Stethoscope: Spirit professional-grade dual-head suspension stethoscope for collecting heart sounds from subjects.
Microphone: JPB lavalier Type-C microphone. Inserted into a chest piece of a stethoscope tubing to record heart sounds through the chest piece of the stethoscope.
Fingertip Pulse Oximeter: Pulse Oximeter clip-on device for measuring post-exercise heart rate for reference.
Sphygmomanometer: Rossmax electronic blood pressure monitor CF175f (with Mandarin and Taiwanese language voice features). Used for measuring blood pressure for data labeling.

### (II) Development Environment:

1. Programming Language: Python
2. Model Training and Data Processing:
   (1) Keras: An open-source neural network library, also an open high-level deep learning library, built as a high-level API on TensorFlow.
   (2) TensorFlow: An open-source deep learning framework provided by Google, supporting various deep learning algorithms.
   (3) NumPy: An extension library for Python, primarily dealing with multi-dimensional array operations, often used for large data processing.
   (4) OS: A module built into Python related to the file system, which can be used to manipulate directories and files.
3. Image Generation and Processing:
   (1) Matplotlib.Pyplot: A sub-library of Matplotlib, it is a commonly used plotting module for creating 2D charts.
   (2) PIL.Image: PIL (Pillow) is an image processing package in Python, with the Image module containing almost all the basic image manipulation functions.

### (III) Training Environment: Google Colab

Google Colab is an online cloud-based Python execution environment based on Jupyter Notebook. It provides free GPU computing power and can connect to Google Cloud to access data in the cloud. With its collaborative editing feature, Google Colab was chosen for use.

### (IV) Audio Editing Software: Audacity

This software is an audio editing program, offering functions such as audio clipping, file conversion, normalization, noise reduction, etc.

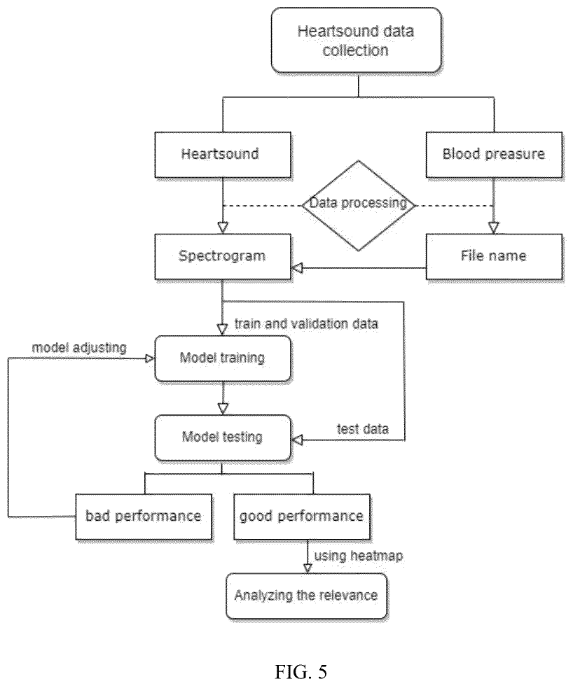

Referring to FIG. 4, it is illustration of a training procedure of a network processor. The training procedure comprises the following step (1) to step (4). A flowchart of the training procedure is shown in FIG. 5.

### (1) Recording (First Pre-Training Step; Heart Sound Data Collection)

### 1A. Ethical Statements

In compliance with domestic human research law, medical law, and other relevant regulations, the “informed consent form” for subjects is provided. This form details the study's purpose and content, the benefits and potential risks for the subject, and requires the subject's consent. Should a participant wish to withdraw, the recording ceases immediately, and their personal information is deleted. All data is coded to ensure de-linking. Furthermore, instructors meet the requirement of having at least nine hours of medical ethics study in the past six years.

The recording includes, for example, 41 subjects. Each subject records 20-30 seconds of heart sounds, which will contain 30-50 heartbeats, totaling 877 data files.

### 1B. Data Collection

Data is collected in a soundproof classroom to ensure its quality. The classroom has a first position, a second position and a third position. Subjects initially read the informed consent forms thoroughly at the first position. If they agree to participate, they move to the second position to have their blood pressure measured. Finally, heart sounds are recorded at the third position.

To fulfill the needs, data is collected in sitting still status (B-1) and collected after exercising (1B-2):

### (1B-1) Sitting Still Status:

a. Measure blood pressure after a subject sits on a comfortable chair with a backrest on the second position for about 5 minutes. During the measuring process, the subject must keep silent and is not allowed to view the value measured by the machine.

b. The subject moves to the third position, and uses a module to press on the area between the second and third ribs at the left edge of the sternum.

The module, also the module 302 (FIG. 3), the module 202 (FIG. 2) or the module 102 (FIG. 1), may comprise a chest piece 302a of a stethoscope, a microphone 302c and a catheter 302b between the microphone 302c and the chest piece 302a of the stethoscope. A computer, may also be the network processor 304, is being connected with the module 302 to record heart sounds from the subject. The computer 304 may comprise an output interface 308. The output interface 308, may also be the output interface 108, an output interface 208 (FIG. 2), or the output interface 308 (FIG. 3), is for displaying a blood pressure level to detect hypertension.

### (1B-2) After Exercising:

a. After a subject exercises and reaches a heart rate of 120 beats per minute, the subject's blood pressure is measured by a machine at the second position. During the measuring process, the subject must keep silent and is not allowed to view the value measured by the machine at the second position.

b. The subject moves to the third position and uses the module to press on the area between the second and third ribs at the left edge of the sternum.

A computer, may also be the network processor 304, is being connected with the module 302 to record heart sounds from the subject. An output interface, may also be the output interface 108, an output interface 208 (FIG. 2), or an output interface 308 (FIG. 3), is for displaying a blood pressure level to distinguish long-term hypertension and exercise-induced transient hypertension.

There are 41 subjects in this first pre-training step, 14 people with hypertension, and 7 people measured after exercising (B-2), see also Table 2. We record about 30 seconds of heart sound for each person, which includes 20˜30 beats. The total amount of data is 877 files.

### (2) Data Processing (Second Pre-Training Step; Transforming Audio Signals into Time-Frequency Spectrograms)

2A. Retrieve and denoise heart sounds using Audacity, as shown in FIG. 4. To be more precise, based on the causes of hypertension, the heart sound audio files are divided into the first and second heart sounds (S1+S2) and the second heart sound (S2) alone. The process of manually extracting heart sound audio files using the Audacity program is as follows:
(1) Import the heart sound audio file into the Audacity application, extract the noise characteristics between the periods of silence among heart sounds, and then use the extracted noise characteristics for noise suppression of the audio file.
(2) Extract the S1+S2 from the heart sound audio file and save it as a .wav file, then separately extract the S2 alone and save it as a .wav file.

2B. Use STFT to transform audio signal into time-frequency spectrograms 320 (FIG. 4). The extracted audio files are processed with short-time Fourier transform to generate time-frequency spectrograms 320 in batch using a program. The conversion process for the time-frequency spectrograms 320 is as follows:
(1) Use a loop to apply the Short-time Fourier Transform (STFT), a discrete Fourier transform (Fast Fourier Transform, FFT), provided by the fastai_audio library, to convert the .wav files into time-frequency spectrograms.
(2) After conversion, it was found that the images are automatically framed with a white border. To avoid the white border affecting the training results, use plt.axis (‘off’), bbox_inches=‘tight’, and pad_inches=0 to remove the white border.
(3) Further convert the time-frequency spectrograms 320 into 2172234.png files for storage, to be used as the training dataset for a deep learning system.

### 2C. Rename the File Using the Format:

The format for the audio file names is as follows: PIDOS1. Here, ‘P’ stands for the participant number, ‘D’ for the hypertension grade, and ‘S’ for the sequence of the extracted audio file. By naming the files in this manner, the attributes of the data can be directly understood from the file name.

The classification of blood pressure is based on the latest high blood pressure standards (The New Blood Pressure Guideline) published by the American Heart Association (AHA) in 2019. The levels of blood pressure according to the standard of the AHA, are shown in Table 1.

TABLE 1
Levels of blood pressure.

| Blood Pressure | | | |
|---|---|---|---|
| Category | Systolic (mmHg) | | Diastolic (mmHg) |
| Normal | <120 | And | <80 |
| Elevated | 120-129 | And | <80 |
| Stage 1 Hypertension | 130-139 | Or | 80-90 |
| Stage 2 Hypertension | >140 | Or | >90 |
| Hypertensive Crisis | >180 | And/Or | >120 |

D0 represents normal blood pressure; D1 represents elevated blood pressure; D2 represents stage 1 hypertension; D3 represents stage 2 hypertension as well as a hypertensive crisis.

TABLE 2
Composition of database

| | | | Stage 1 | |
|---|---|---|---|---|
| | | | Hypertension | Stage 2 |
| | Normal | Elevated | (SBP: 130-139 | Hypertension |
| | (SBP < 120 or | (SBP: 120-129 or | and | (SBP > 140 and |
| Status | DBP < 80) | DBP < 80) | DBP: 80-90) | DBP > 90) |
| Sitting Still | 20 subjects | 9 subjects | 0 subject | 5 subjects |
| (1B-1) | | | | |

### (3) Model Training

### 3A. CNN

We use Convolution Neural Network (CNN) in this step. CNN is an algorithm in deep learning, whose structure resembles the connections of neurons in the human brain. The convolution layers, pooling layers, and fully connected layers in the model can extract features in the images and make predictions.

The model training is conducted using a CNN model architecture, and the detailed steps are as follows:

Step 3A-1. Read the time-frequency spectrograms data and use the file names to create the correct database (Y).
For example, to create databases for different blood pressure classifications, first find the “D” in the read file names, then determine the number that follows as 0, 1, 2, or 3.
The method of creating the blood pressure classification matrix is as follows: if the number after “D” is 0, then enter [1,0,0,0] into Y; if the number after “D” is 1, then enter [0,1,0,0] into Y; if the number after “D” is 2, then enter [0,0,1,0] into Y; if the number after “D” is 3, then enter [0,0,0,1] into Y.
In the classification of hypertension caused by exercise and long-term hypertension, [1,0] represents long-term hypertension (0), and [0,1] represents exercise-induced hypertension (1).

### Step 3A-2. Data Splitting and Shuffling.

To avoid bias in the model due to the order of the data during training, we use the random.permutation function from NumPy to shuffle all the data. After shuffling, we use the split function in NumPy to divide the data into three parts for train, validation, and test, based on a 6:2:2 ratio.
Considering that this model may be used for medical purposes, to ensure that the model can truly identify data on hypertension rather than making judgments through learning individual differences between test subjects, we also adopt a person-wise shuffling approach for data that is closer to real-world applications.
First, we use random.sample to randomly select a data entry from the D0, D1, D2, D3 categories. Then, we read the participant number information from the filename of the selected data, and in subsequent program reads, we store the files with this number separately in the array for test data. We use random.permutation to shuffle the arrays for both the test data and the remaining data separately. Finally, we use split to divide the remaining data array based on a 3:1 ratio into two parts for train and validation. After shuffling the data in this manner, the model will not encounter any data from the test database's participants during training, thus avoiding the concern that the model is learning personal characteristics rather than blood pressure level features.

Table 3 shows the data splitting and its function

| | Participation | |
|---|---|---|
| | in training | |
| | or not. | Function |
| train | Yes | Conduct model training. |
| validation | Yes | Adjust the model's accuracy after each epoch |
| | | of training. |
| test | No | Reserved as the basis for evaluating the final |
| | | training results of the model. |

### Step 3A-3 Model Structure

A multi-layer CNN network structure model is constructed using the Keras neural network library. Each layer structure consists of one convolution followed by one max pooling. To prevent overfitting, we employed two solutions: the first is to add a dropout layer before the dense layer, and the second is to insert Batch Normalization between the convolutional layers. The compile function is then used to define the loss as ‘categorical_crossentropy’, the optimizer as ‘adam’, and the metrics as ‘accuracy’. Furthermore, we use ReduceLROnPlateau to gradually decrease the learning rate.

TABLE 4
Model structure in Step 3A-3 (A Four-
Layer Model Architecture Diagram)

| Layer (type) | Output Shape | Param # |
|---|---|---|
| input_layer (InputLayer) | [(None, 217, 223, 4)] | 0 |
| conv1(Conv2D) | (None, 109, 112, 32) | 1152 |
| batch_normalization_4 | (None, 109, 112, 32) | 128 |
| (BatchNormalization) | | |
| activation_4 (Activation) | (None, 109, 11232) | 0 |
| conv2(Conv2D) | (None, 55, 56, 64) | 18432 |
| batch_normalization_5 | (None, 55, 56, 64) | 256 |
| hNormalization) | | |
| activation_5 (Activation) | (None, 55, 56, 64) | 0 |
| conv3(Conv2D) | (None, 28, 28, 128) | 73728 |
| batch_normalization_6 | (None, 28, 28, 128) | 512 |
| hNormalization | | |
| activation_6 (Activation) | (None, 28, 28, 128) | 0 |
| conv4(Conv2D) | (None, 14/4,256) | 294912 |
| batch_normalization_7 | (None, 1414256) | 1024 |
| (BatchNormalization) | | |
| activation_7 (Activation) | (None, 14, 14, 256) | 0 |
| global_average_poolingzd_1 | (None, 256) | 0 |
| (GlobalAveragePooling2D) | | |
| reshape_1(Reshape) | (None, 1, 256) | 0 |
| dropout_1(Dropout) | (None, 1, 256) | 0 |
| output_layer(Dense) | (None, 1, 4) | 1028 |

Total params: 393,172
Trainable params: 390,212
Non-trainable params: 960

### Model Training Loss and Accuracy Graphs

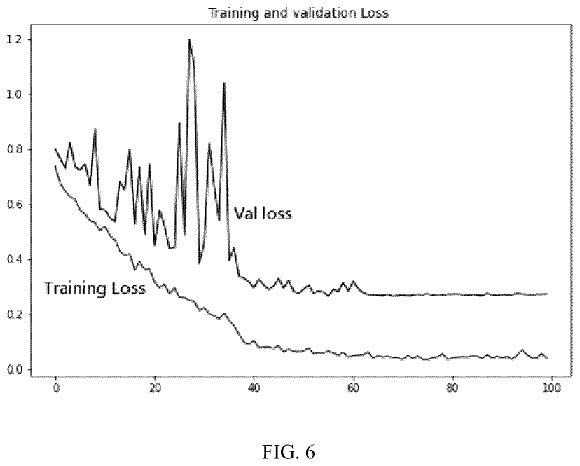

FIG. 6 is a Loss graph for four-layer model training with S2 data. In FIG. 6, the training loss exhibits a significant decrease in the first 40 epochs, after which the rate of decline becomes very gradual. The validation loss shows considerable fluctuation in the first 40 epochs, especially between the 20th to 40th epochs. However, the fluctuations gradually smooth out between the 40th to 60th epochs and nearly flatten after the 60th epoch. Considering both lines, although the validation loss remains somewhat higher than the training loss towards the end, it still converges to around 0.3, while the training loss converges to around 0.05. Additionally, the converging trend of both lines is quite apparent, leading us to conclude that this is a successful model training.

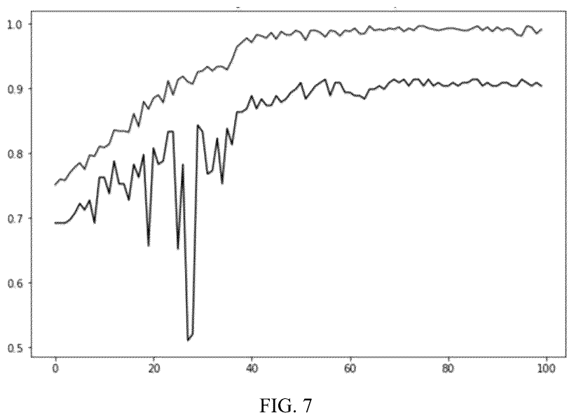

FIG. 7 presents an accuracy graph with a lower curve and an upper curve, representing the training of a four-layer model using S2 data. The horizontal axis denotes the epoch, while the vertical axis measures the accuracy in percentages, up to 100%. In FIG. 7, the upper curve indicates that the training accuracy progressively increases during the first 40 epochs and then levels off after the 40th epoch. The lower curve shows that the validation accuracy experiences substantial fluctuations within the initial 40 epochs, most notably between the 20th and 30th epochs. These fluctuations gradually subside between the 30th and 60th epochs and become minimal after the 60th epoch. Observing both curves together, it's noticeable that even though the validation accuracy doesn't match the high level of the training accuracy towards the end, it still reaches a commendable level of about 90%, while the training accuracy achieves a substantial rate of approximately 98%. Hence, this graph demonstrates positive training results.

### Step 3A-4 Parameters

The model is trained using Keras's model.fit and predicts the outcome of the validation dataset to adjust that training iteration. A single run of the model's training is known as one epoch. In this scientific exhibition, performing 100 epochs of training typically achieves a high accuracy rate.
During the model training process, if the loss between the correct answers in the train database and the predicted values has converged, but the val_loss for the validation database and predicted values continues to diverge, it indicates that the model is overfitting. In this case, training should be stopped, and the model should be readjusted.
If the difference (val_loss) between the validation database and the predicted values also converges, then at the end of training, the model with the smallest loss is recorded. This model is then used to make predictions on the test database to calculate the accuracy of the predictions.

TABLE 5
Parameter form

| | |
|---|---|
| train:validation:test | 6:2:2 |
| input shape | (217, 223, 4) |
| number of filters | 32, 64, 128, 256 |
| filter size | (3, 3) |
| stride | (2, 2) |
| padding | same |
| activation function | relu, softmax |
| pooling | average pooling |
| learning rate | 0.001, reduce learning rate |
| optimizer | Adam |
| epoch | 100 |

### (4) Model Evaluation

### Step 4A

To fully grasp the condition of model training and to determine any bias, this scientific exhibition uses the Confusion Matrix and associated performance metrics (Accuracy, Precision, Recall, and F1 score) to evaluate the model. Additionally, a heatmap is used to visualize the model's decision-making basis, providing deeper insight into its reasoning process.
Here are introductions to the aforementioned indicators:

### Confusion Matrix:

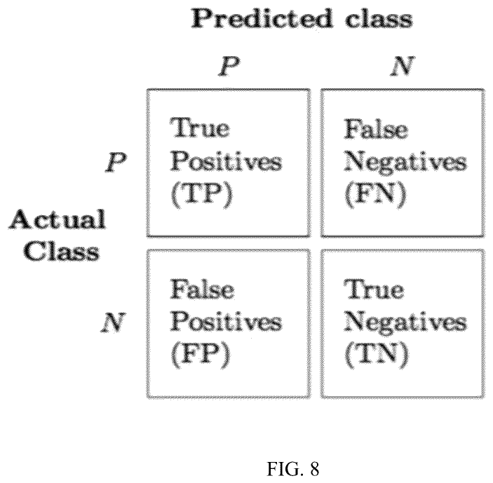

This matrix serves as an analytical table to assess the results of a model, with the vertical axis representing actual labels (Actual Class) and the horizontal axis representing predicted results (Predicted Class).
FIG. 8 is a schematic diagram of the confusion matrix. In FIG. 8, for a binary model (Positive, P, and Negative, N), if both the actual and predicted outcomes are P, then this result is classified as True Positives (TP); if the actual outcome is P but predicted as N, then it is classified as False Negative (FN); if the actual outcome is N but predicted as P, then it is classified as False Positive (FP); if both the actual and predicted outcomes are N, then this result is classified as True Negatives (TN).
After filling in the outcomes of the model's decisions into the chart, the values of TP, FN, FP, and TN can be used to further calculate the model's Accuracy, Precision, Recall, and F1 score.

The calculation is as the following:
a. Accuracy=(TP+TN)/(TP+FP+FN+TN) This indicator represents the percentage of correct judgments in all cases.
b. Precision=TP/(TP+FP) This indicator represents the percentage of all positive samples that are truly positive.
c. Recall=TP/(TP+FN) This indicator represents the number of successful judgments when the facts are true.
d. F1 score=2/((1/Precision)+ (1/Recall)) This is the harmonic mean of the precision and recall. This metric takes both precision and recall into account and can be used as a comprehensive measure.
   Step 4b. Heatmap
When applied to CNN models, Heatmap can visualize the judgment basis of the model layer. The color function used in our study is jet. The more focused part in model judgment, the redder the superimposed color will be. On the contrary, the color will be bluer.

### (5) Result

When using S2 as the database compared to S1+S2, there is a smaller gap between training loss and validation loss, indicating better model convergence. The following is a detailed analysis and comparison of 1. model evaluation metrics, 2. the confusion matrix, and 3. the heatmap.
1. Comparing Results of Model Trained by Different Dataset (S1+S2/S2)
1-1 Purpose: To improve accuracy by removing irrelevant data
1-2 Result: We can see from Table 7 that the model trained by only S2 data performs better than the model trained by S1+S2 data. Therefore, we can conclude from the result that features of blood pressure can be reflected in time-frequency spectrograms and it also confirms the statement that S2 is more related to hypertension.

TABLE 7
Comparison of model metrics for training on different databases.

| S1 + S2 | S2 |
|---|---|
| Test Accuracy: 0.8148 | Test Accuracy: 0.8889 |
| Precision: 0.6639 | Precision: 0.7901 |
| Recall: 0.8148 | Recall: 0.8889 |
| F1 Score: 0.7317 | F1 Score: 0.8366 |

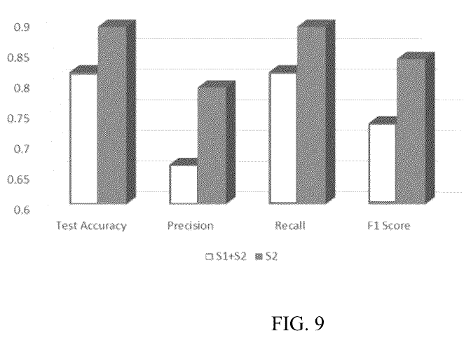

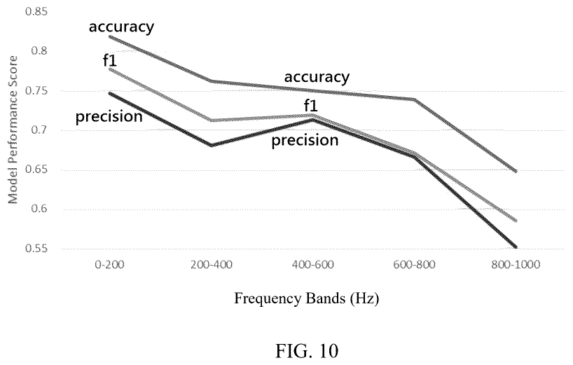

A diagram that presents the performance of models trained with S1+S2 Dataset and S2 Dataset is shown in FIG. 9.
1-3 Heatmap: Table 8. Comprises heatmaps of models trained by different datasets. Fromm Table 8, it is learned that frequency bands of 0-200 Hz and 400-600 Hz are more related to hypertension. Also, it can be inferred that the features of blood pressure reflected in time-frequency spectrograms are obvious.
2.Relationship between Frequency Band and Model Performance
2-1 Purpose: We can see from the previous heatmaps that the colored patterns on it are mostly horizontal, so we designed this experiment, excepting to find out the important frequency bands from the model's performance.
2-2 Result: We can learn from the result that the models trained by lower frequency bands have higher accuracy. Also, we can see that the frequency band of 400-600 Hz has a slightly better performance than 200-400 Hz, which is different from the original declining trend. Therefore, we assume that the frequency bands of 0-200 Hz and 400-600 Hz are the most crucial part. See also FIG. 10, which shows a diagram that presents the performance of models trained by different frequency bands.
3. Distinguishing between Exercise-induced Hypertension and Long-term Hypertension
   (1) Purpose: The cause of exercise-induced hypertension and long-term hypertension aren't the same, so we hope to know if spectrograms can show the differences.
   (2) Result: Although there were only about 160 data in this experiment, the results of model training were still quite good. There were 33 testing data, about 20% of the total data, that weren't exposed to the model during training, but only 2 of them were incorrect. Although the current database is small, it has been proven that exercise-induced hypertension and long-term hypertension can be distinguished by CNN from spectrograms.

TABLE 5
Evaluation indicators of the model.

| Test Accuracy | Precision | Recall | F1 score |
|---|---|---|---|
| 0.9394 | 0.9449 | 0.9394 | 0.9383 |

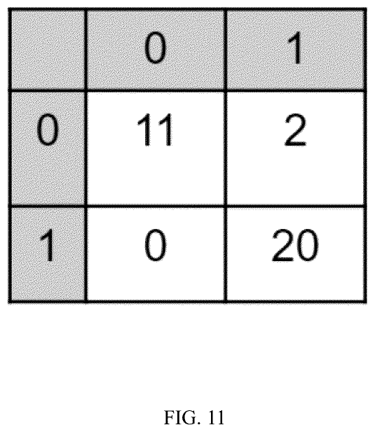

FIG. 11 is the confusion matrix for the model training distinguishing between post-exercise hypertension and long-term hypertension. The matrix includes the following data points. The number 11 in the top left cell indicates the True Positives (TP), which means the model correctly predicted 11 instances of the actual class ‘0’, which could represent post-exercise hypertension. The number 2 in the top right cell signifies the False Negatives (FN), where the model incorrectly predicted 2 instances of the actual class ‘0’ as class ‘l’, meaning that two cases of post-exercise hypertension were incorrectly identified as chronic hypertension. The number 0 in the bottom left cell stands for False Positives (FP), showing that there were no instances where the actual class ‘l’, which could represent chronic hypertension, was incorrectly identified as class ‘0’. The number 20 in the bottom right cell indicates the True Negatives (TN), which means the model correctly predicted 20 instances of the actual class ‘l’, identifying them accurately as chronic hypertension.

The numbers ‘0’ and ‘l’ on the top of the matrix represent the predicted classes by the model, with ‘0’ possibly denoting post-exercise hypertension and ‘l’ denoting chronic hypertension. Similarly, the ‘0’ and ‘l’ on the left side of the matrix represent the actual classes of the conditions being predicted. The confusion matrix in FIG. 11 thus demonstrates that the model has high predictive accuracy, with a total of 31 correct predictions out of 33 (11 TP+20 TN), and only 2 incorrect predictions (2 FN), evidencing the model's excellent training performance. Based on the results of the confusion matrix in FIG. 11, the model's predictions are very accurate, with only 2 data points predicted incorrectly, indicating excellent training results.

### (6) Discussions

1. Using dataset consisting of only S2 data leads to better results.

We learn that S1 happens in the beginning of the systole phase, and is caused by the closing of mitral and tricuspid valves. S1 is easily affected by the size and thickness of hearts. S2, on the other hand, happens in the beginning of the diastole phase, and is caused by the halting of the aortic and pulmonary valves leaflets. What's more, S2 is suggested to be more related to hypertension. As we can see from the results, the model trained by only S2 data performs better in all aspects. We suspect that it is because the model can focus on features that are more important after we eliminate S1 data. Our results and paper lead to the same conclusion that S2 is more important when detecting hypertension.

2. 0-200 and 400-600 Hz are the important frequency bands related to hypertension.

From the heatmap of experiment 1, we noticed that the patterns are mostly horizontal. Since the y axis of spectrograms represents frequency, we assumed that the image features the model extracted might be associated with certain frequency bands. Therefore, in experiment 2, we use spectrograms of different frequency bands to train the model and compare the results. We discovered that models trained by spectrograms of 0-200 and 400-600 Hz have better performance.

3. Exercise-induced hypertension can be distinguished from long-term hypertension.
Although the database size of this experiment is relatively small, we can see from the F1 score that the model still performs well, which indicates that structural differences have corresponding image features on spectrograms and can be learned by the model. The ability to detect structural differences broadens the application of this study, and the same method may be able to apply to other cardiovascular diseases.

### (7) Conclusions

1. A CNN model on heart sound can be trained to accurately measure blood pressure.

The model can detect levels of blood pressure accurately. The results show the high feasibility of using CNN model on heart sounds to detect hypertension.

2. Furthermore, exercise-induced high blood pressure measurements can be detected to prevent false alarms.

The model successfully distinguished exercise-induced hypertension and long-term hypertension. Since long-term hypertension results in structural differences, we can know that there are corresponding image features of structural differences on spectrograms, and these image features can be extracted and learned by CNN.

3. Results from this research pave the way for non-pressurized wearable devices that can constantly monitor blood pressure for early detection of hypertension.

Heart sounds are easy to obtain, and can be collected in a non-pressurized way. What's more, by using our model, we can continuously monitor users' level of blood pressure and prevent misjudgments of hypertension when one is only exercising. In conclusion, our study has the potential to become a wearable device and contribute to telemedicine.

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An apparatus for quick detection, comprising:
a module for capturing data of a heart sound;
a device for transforming the data into a plurality of time-frequency spectrograms having a plurality of image features; and
a network processor trained to analyze the image features of the time-frequency spectrograms, for giving a blood pressure level.

2. The apparatus of claim 1, further comprising an output interface for displaying the blood pressure level.

3. An apparatus for quick detection, comprising:
a non-pressurized module for capturing data of a second heart sound;
a device for transforming the data into a plurality of time-frequency spectrograms having a plurality of image features; and
a network processor equipped with a convolutional neural network trained to analyze the image features of the time-frequency spectrograms, by focusing on the second heart sound, for detecting hypertension.

4. The apparatus of claim 3, wherein each of the time-frequency spectrograms comprises a first frequency band and a second frequency band.

5. The apparatus of claim 4, wherein the network processor trained to analyze the image features of the time-frequency spectrograms within the first frequency band and the second frequency band.

6. The apparatus of claim 5, wherein the first frequency band is about 0 Hz to about 200 Hz.

7. The apparatus of claim 5, wherein the second frequency band is about 400 Hz to about 600 Hz.

8. The apparatus of claim 3, wherein the non-pressurized module captures the data by recording the second heart sound for about 20 to about 30 seconds.

9. The apparatus of claim 3, wherein the second heart sound is recorded from an individual who has been sitting down for a plurality of minutes.

10. The apparatus of claim 9, wherein the second heart sound is recorded from the individual who has been sitting down for about five minutes.

11. The apparatus of claim 9, wherein the second heart sound is recorded from the individual who has exercised.

12. The apparatus of claim 11, wherein the second heart sound is recorded from the individual who has exercised to have a heart rate of about 120 beats per minute.

13. An apparatus for quick detection, comprising:
a module for capturing data of a heart sound;
a device for transforming the data into a plurality of time-frequency spectrograms having a plurality of image features, wherein each of the time-frequency spectrograms comprises a frequency band; and
a network processor, trained to identify the time-frequency spectrograms within the frequency band, to distinguish long-term hypertension and exercise-induced transient hypertension.

14. The apparatus of claim 13, wherein the frequency band is about 0 Hz to about 200 Hz.

15. The apparatus of claim 13, wherein the frequency band is about 400 Hz to about 600 Hz.

16. The apparatus of claim 13, wherein the module captures the data by recording the heart sound.

17. The apparatus of claim 13, wherein the module captures the data by recording the heart sound for about 20 to about 30 seconds.

18. The apparatus of claim 13, wherein the heart sound is recorded from an individual who has been sitting down for a plurality of minutes.

19. The apparatus of claim 18, wherein the heart sound is recorded from the individual who has exercised.

20. The apparatus of claim 19, wherein the heart sound is recorded from the individual who has exercised to have a heart rate of about 120 beats per minute.
